(19) United States
(12) Patent Application Publication
(10) Pub. No.: US 2009/0134066 A1
(43) Pub. Date: May 28, 2009

(54) Method for producing alkyl-aromatic compounds by direct alkylation of aromatic hydrocarbons with alkanes

(75) Inventors: Nils Bottke, Mannheim (DE); Michael Triller, Mannheim (DE); Ulrich Muller, Neustadt (DE); Rolf Pinkos, Bad Durkheim (DE); Thomas Heidemann, Viernheim (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF AKTIENGESELLSCHAFT, LUDWIGSHAFEN (DE)

(21) Appl. No.: 11/659,422

(22) PCT Filed: Aug. 4, 2005
(86) PCT No.: PCT/EP2005/008465

§ 371 Date: Oct. 24, 2007

(30) Foreign Application Priority Data
Aug. 5, 2004 (DE) 10 2004 038 108.9

(51) Int. Cl. C10G 35/06; C07C 2/58

(52) U.S. Cl. 208/141; 585/468

(57) ABSTRACT

The present invention relates to a process for preparing alkylaromatics by reacting aromatic compounds with C₁-C₁₄-alkanes in the presence of a heterogeneous catalyst, which comprises using as the catalyst a crystalline, micro- and/or mesoporous solid comprising silicon and at least one father element selected from the group consisting of the transition metals and the main group elements gallium and tin, and activating said catalysts by a reducing pretreatment. Furthermore, the present invention relates to a process for preparing alkylarylsulfonates by sulfonating and neutralizing the alkyl aromatic compounds.

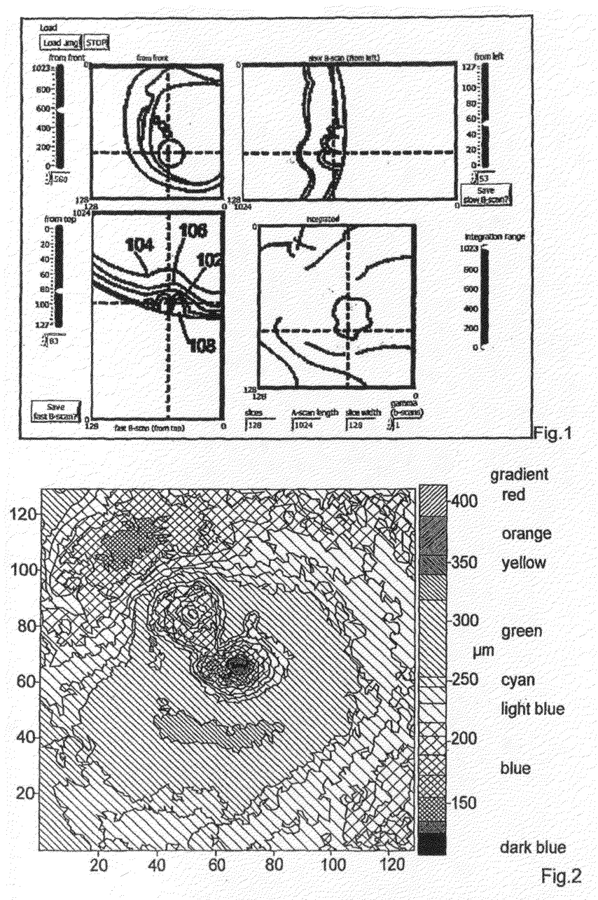

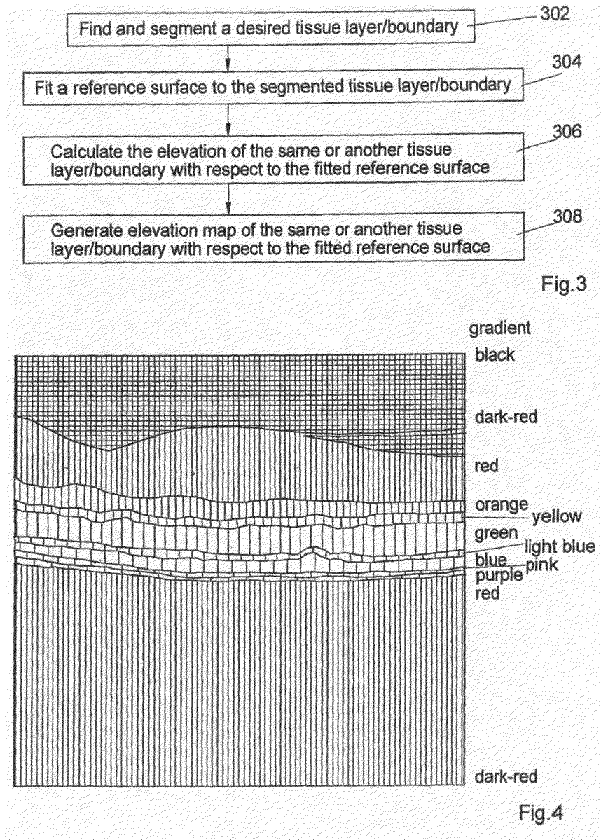

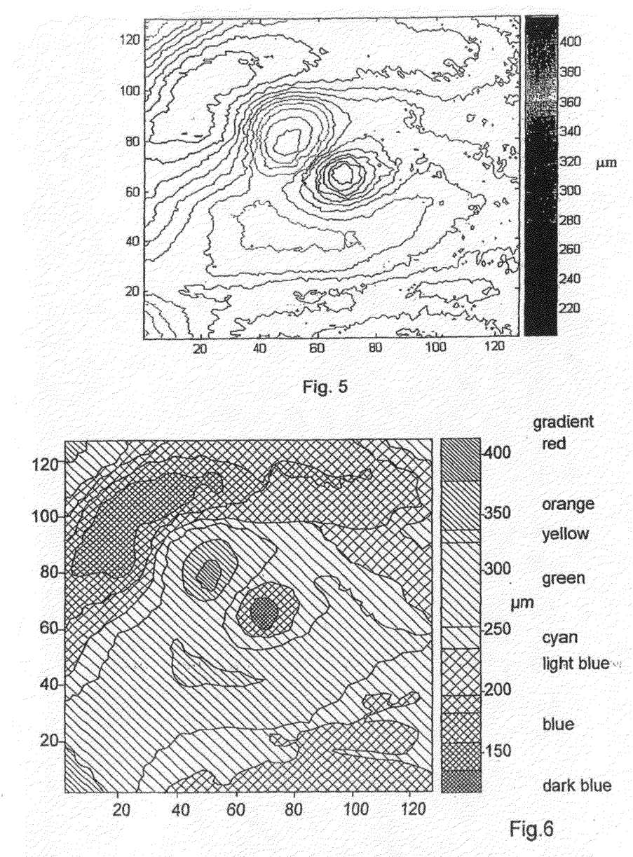

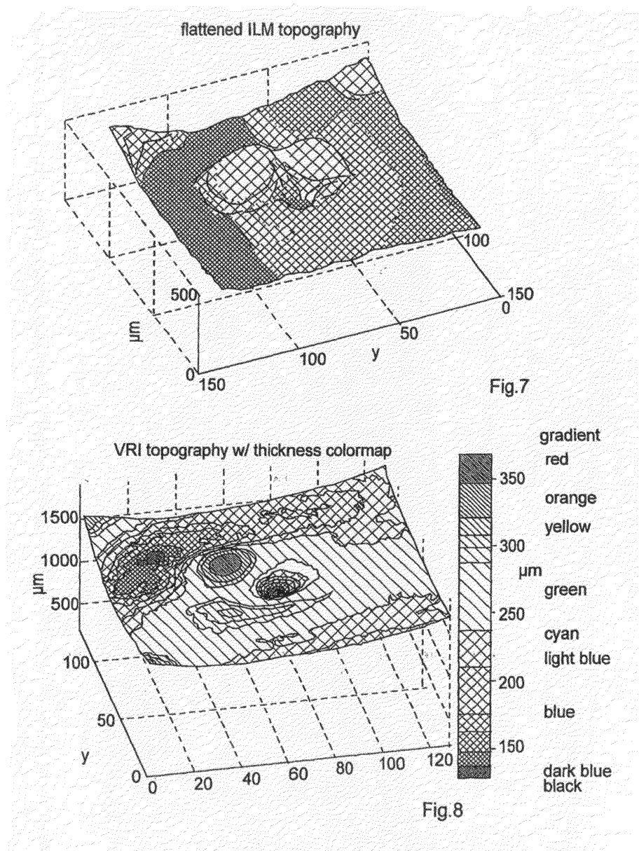

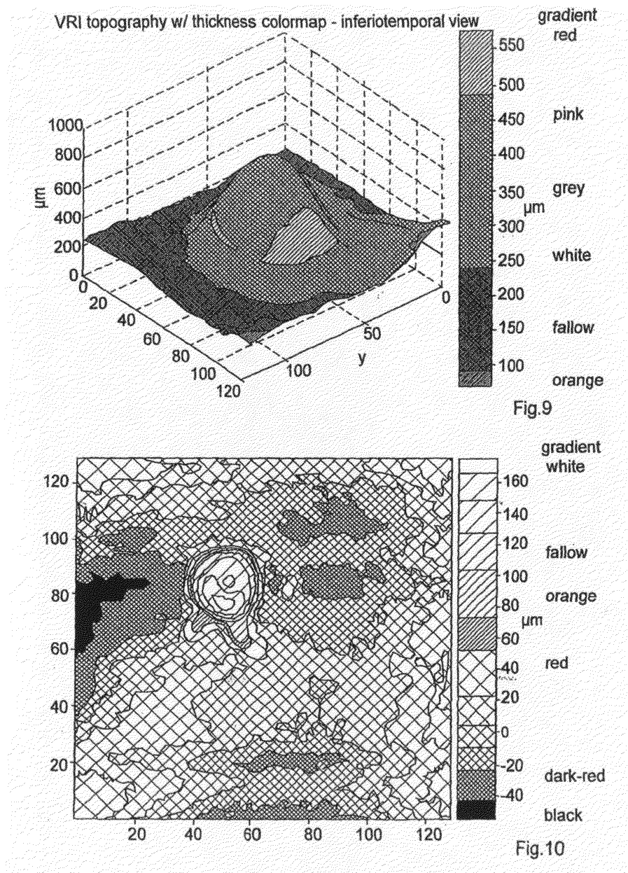

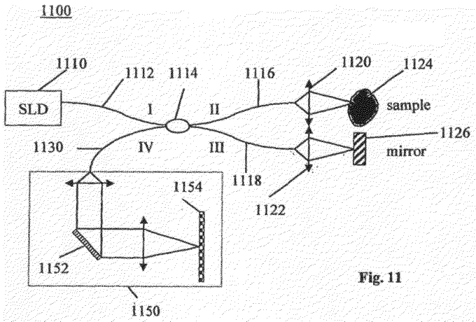

The present invention relates to a process for preparing alkylaromatics by reacting aromatic compounds with C₁-C₁₄-alkanes in the presence of a heterogeneous, crystalline, micro- and/or mesoporous catalyst which has been activated by a reducing pretreatment, and to a process for preparing alkylarylsulfonates by sulfonating and neutralizing these alkylaromatics.

For the preparation of alkylaromatics, there are various processes employed in industry which all proceed via an activation of the alkane in a separate process stage. This activation can be effected, for example, by dehydrogenating to the corresponding alkene or by chlorinating to the corresponding chloroalkane.

Processes are also known for obtaining alkylaromatics by direct reaction of alkanes with aromatic compounds.

U.S. Pat. No. 3,109,038 discloses a process for preparing alkylaromatics by reacting C₂-C₁₀-alkanes with aromatic compounds in the presence of a catalyst which comprises transition group metals on a support composed of oxides of aluminum, silicon and/or boron. Preference is given to reacting the aromatic compounds with ethane, propane and/or butane. The catalyst is activated by reductive pretreatment before the reaction of the aromatic compound with the alkanes.

U.S. Pat. No. 4,899,008 discloses a process in which C₂-C₄-alkanes are reacted with monocyclic aromatics to give the corresponding alkylaromatics.

U.S. Pat. No. 5,900,520 likewise describes a process for preparing alkylaromatics, in which C₁-C₁₄-alkanes, preferably C₁-C₈-alkanes, are reacted with aromatic compounds in the presence of an alkylation catalyst which has a particular X-ray diffraction pattern. The catalyst used is not reductively pretreated before the reaction.

In WO 99/59942, C₁₅-C₂₂-alkanes are reacted, in the presence of molecular sieves which have been doped with various metals, with aromatic compounds to give the corresponding alkylaromatics. The catalyst is activated by a reductive pretreatment before the reaction.

Alkylbenzenesulfonates (ABS) are used in surfactants in laundry detergents and cleaning compositions. After such surfactants based on tetrapropylenebenzenesulfonate had been used at first, but had poor biodegradability, substantially linear alkylbenzenesulfonates (LAS) were prepared and used in the subsequent time.

It is an object of the present invention to provide a process for preparing alkylaromatics from aromatic compounds and alkanes without separately activating the alkanes, in which the catalyst used is activated by a reducing pretreatment. It is a further object of the present invention to provide a process by which it is possible to prepare alkylarylsulfonates starting from these alkylaromatics by sulfonation and subsequent neutralization.

The achievement of the object starts from a process for preparing alkylaromatics by reacting aromatic compounds with C₁-C₁₄-alkanes in the presence of a heterogeneous catalyst.

The process according to the invention comprises using as the catalyst a crystalline, micro- and/or mesoporous solid comprising silicon and at least one further element selected from the group consisting of the transition metals and the main group elements gallium and tin, and activating said catalysts by a reducing pretreatment.

The present invention thus relates to a process for preparing alkylaromatics by reacting aromatic compounds with C₁-C₁₄-alkanes in the presence of a heterogeneous catalyst, wherein the catalyst used is a crystalline, micro- and/or mesoporous solid comprising silicon and at least one further element selected from the group consisting of the transition metals and the main group elements gallium and tin, and the catalyst is activated by a reducing pretreatment.

In the process according to the invention, it is possible to use mono- or polycyclic aromatic compounds. It is possible to use optionally substituted benzene, optionally substituted naphthalene, optionally substituted indene, optionally substituted fluorene, optionally substituted anthracene, optionally substituted phenanthrene or optionally substituted tetracene. In a preferred embodiment, monocyclic, aromatic compounds are used in the process according to the invention.

Substituents of these aromatic compounds which can be used in the process according to the invention may be linear or branched, saturated or unsaturated hydrocarbon radicals having from 1 to 25 carbon atoms which may optionally be substituted by at least one functional group, such as the hydroxyl, amino, imino, imido, keto, ether, aldehyde or carboxyl group. The substituents are preferably selected from the group consisting of linear or branched alkyl radicals having from 1 to 10 carbon atoms, and the substituents are more preferably methyl, ethyl, propyl, butyl, pentyl or hexyl radicals.

Particular preference is given to using a compound selected from the group consisting of benzene, toluene, ethylbenzene and the isomers of xylene.

Very particular preference is given to using a compound selected from the group consisting of benzene, toluene and ethylbenzene. Special preference is given in the process according to the invention to using benzene.

The aromatic compounds which can be used in the process according to the invention may be prepared or obtained by methods known to those skilled in the art. Examples include thermal or catalytic extraction from coal or mineral oil, azeotropic distillation from reformate and pyrolysis benzine, extraction, inter alia.

It is also possible in the process according to the invention to use mixtures of the aforementioned aromatic compounds.

In the process according to the invention, C₁-C₁₄-alkanes can be used. In a preferred embodiment, C₉-C₁₄-alkanes are used in the process according to the invention. Particular preference is given to using C₁₀-C₁₃-alkanes in the process according to the invention. Very particular preference is given to using a C₁₂-alkane, dodecane, in the process according to the invention.

Alkanes which can be used in accordance with the invention may be linear or branched. Preference is given to using alkanes which have a degree of branching less than or equal to 1. Particular preference is given to using linear alkanes.

The degree of branching of an alkane describes the average number of branches of the carbon chain per molecule. A degree of branching of 1 means that each molecule of the alkane present is on average singly branched.

In the process according to the present invention, it is possible to use either alkanes having a uniform carbon number or mixtures of alkanes having a different number of carbons. It is also possible to use mixtures of different isomers of alkanes having the same carbon number.

The alkanes or mixtures of alkanes which can be used in the process according to the invention may be obtained by processes known to those skilled in the art. Examples include distillation and extraction of mineral oil and natural gas, coal hydrogenation and Fischer-Tropsch synthesis, LPG (liquefied petroleum gas), LNG (liquefied natural gas) and GTL (gas-to-liquids).

The process according to the invention is carried out in the presence of a heterogeneous catalyst. The catalyst which can be used in the process according to the invention is a crystalline, micro- and/or mesoporous solid.

In the present application, crystalline means that the individual molecules or atoms of the catalyst are arranged in a regular long-range order in a lattice structure. Preferably more than 80% by weight, more preferably more than 90% by weight, of the catalyst used is in crystalline form.

According to IUPAC, the pore radii of porous solids are divided as follows: microporous refers to solids having average pore radii up to 2 nm; mesoporous to solids having average pore radii of from 2 to 50 nm.

In the process according to the invention, the catalysts used may be solids which have average pore radii of up to 2 nm and/or of from 2 to 50 nm.

The catalysts used may be of natural or synthetic origin, whose properties can be adjusted to a certain extent by literature methods, as described, for example, in J. Weitkamp and L. Puppe, Catalysis and Zeolites, Fundamentals and Applications, Chapter 3; G. Kühl, Modification of Zeolites, Springer Verlag, Berlin, 1999 (ion exchange, dealumination, dehydroxylation and extraction of lattice aluminum, thermal treatment, steaming, treatment with acids and SiCl₄, blocking of specific, for example external, sites by, for example, silylation, reinsertion of aluminum, treatment with aluminum halides and oxo acids).

In addition, the catalysts may also comprise already used catalyst material or consist of such material which has been regenerated by the customary methods, for example by a recalcination in air, H₂O, CO₂ or inert gas at temperatures greater than 200° C., by washing with H₂O, acids or organic solvents, by steaming or by treatment under reduced pressure at temperatures greater than 200° C.

The catalysts which can be used in the process according to the invention may be used in the form of powders or preferably in the form of shaped bodies such as extrudates, tablets or spall.

For reshaping, it is possible to add to the catalyst from 2 to 80% by weight, based on the composition to be reshaped, of binder. Suitable binders are various aluminas, preferably boehmite, amorphous aluminosilicates, silicon dioxide, preferably highly dispersed silicon dioxide, for example silica sols, mixtures of highly disperse silicon dioxide and highly disperse alumina, highly disperse titanium dioxide, and also clays. The catalyst used in the process according to the invention consequently includes, in addition to the catalytically active component, if appropriate from 2 to 80% by weight of the aforementioned binders. It is also possible that the catalyst used in the process according to the invention does not include any binder.

The catalyst which can be used in the process according to the invention includes at least one further element selected from the group consisting of the transition metals and the main group elements gallium and tin, more preferably selected from groups 6, 7, 8, 9, 10, 11 of the Periodic Table, cerium, zinc, lanthanum and zirconium, most preferably selected from the group consisting of rhenium, iron, ruthenium, cobalt, rhodium, iridium, nickel, palladium, platinum and copper. Depending on the preparation, ammonium, alkali metal, alkaline earth metal ions may also be present.

In a preferred embodiment, the catalyst is at least one zeolite or at least one clay. The catalyst used is more preferably a zeolite.

Suitable catalysts are clays, for example bentonite, kaolinite, montmorillonite, attapulgite, hectorite or sepiolite, and also what are known as pillared clays in conjunction with elements selected from the group consisting of the transition metals and the main group elements gallium and tin.

Additionally suitable are zeolites, especially selected from the group of structural classes consisting of FAU, MOR, BEA, MFI, MEL, TON, MTW, ZBM-11, FER, LTL, MAZ, EPI and GME, most preferably selected from the group consisting of FAU, MOR, BEA and MFI.

In the case of zeolites, the aluminum lattice positions may additionally be partly or fully replaced by an element selected from the group consisting of boron, gallium, iron, titanium, lanthanum, tin and zirconium.

To increase the selectivity, the lifetime and the number of the possible catalyst regenerations, it is additionally possible to undertake various modifications on the catalyst.

The zeolites way be used in the H form or, if appropriate partly, ion-exchanged form, in which case the metal ions are selected preferably from groups 6, 7, 8, 9, 10 and/or 11 of the Periodic Table, gallium, cerium zinc and/or tin, but ammonium, alkali metal and/or alkaline earth metal ions may also be present depending on the preparation. In addition to or independently of a metal ion exchange, the abovementioned metal ions may be applied by impregnation. A partial or full exchange of the lattice aluminum for boron, gallium, iron, titanium, lanthanum, tin and/or zirconium is possible.

In an advantageous embodiment of the catalyst, the catalysts are initially charged as shaped bodies or in powder form in a reactor (for example reaction tube, stirred tank) and treated at from 20 to 100° C. with a metal salt solution of the abovementioned metals, preferably chlorides, nitrates, acetates, oxalates, citrates or mixtures thereof, in a suitable solvent, preferably water. Such an ion exchange may be undertaken, for example, on the hydrogen, ammonium or alkali metal form of the catalysts. Further methods for ion exchange and also for other modifications of zeolites, for example dealumination, are known to those skilled in the art and are described, inter alia, in “Catalysis and Zeolites, Fundamentals and Applications”, Weitkamp, Puppe (Eds.), Springer-Verlag Berlin Heidelberg 1999, pages 81-179.

The metals or mixtures of metals mentioned are present in a concentration of from 0.01 to 25% by weight, preferably from 0.05 to 10% by weight, more preferably from 0.05 to 5% by weight, based in each case on the catalyst.

These elements may be applied to the catalyst by processes known to those skilled in the art. Examples include impregnation of the catalyst support with suitable aqueous or alcoholic solutions of the corresponding elements or suitable compounds, for example with a halide, acetate, oxalate, citrate, nitrate or oxide of the above-described metals. Both the ion exchange and an impregnation may be followed by a drying, if desired a calcination.

The drying may generally be carried out at elevated temperature, preferably at from 50 to 500° C., more preferably at from 100 to 400° C., and a pressure generally below atmospheric pressure, preferably at from 0.1 to 950 mbar, more preferably at from 1 to 500 mbar. The dig may also be carried out at atmospheric pressure.

The calcination may generally be carried out at elevated temperature, preferably at from 100 to 1500° C., more preferably at from 200 to 1000° C. and under conditions otherwise known to those skilled in the art.

In a further method of modifying the catalyst, the heterogeneously catalytic material, shaped or unshaped, is subjected to a treatment with acids, such as nitric acid (HNO₃), hydrochloric acid (HCl), hydrofluoric acid (HF), phosphoric acid (H₃PO₄), sulfuric acid (H₂SO₄), oxalic acid (HO₂C—CO₂H) or mixtures thereof.

A further particular embodiment lies in the acid treatment of the heterogeneous catalysts after they have been shaped with binder. In this treatment, the shaped catalyst is generally treated with a from 3 to 25%, in particular with a from 12 to 20%, acid solution at temperatures between 60 to 80° C. for from 1 to 3 hours, subsequently washed, dried at from 100 to 160° C. and calcined at from 400 to 550° C., The acids used are preferably formic acid, hydrochloric acid, nitric acid and/or sulfuric acid.

Another means of modifying the catalyst is exchange with ammonium salts, for example with NH₄Cl, or with mono-, di- or polyamines. In this modification, the heterogeneous catalyst reshaped with binder is generally treated continuously with from 10 to 25%, preferably approx. 20%, ammonium chloride solution at from 60 to 80° C. for 2 hours, the weight ratio of heterogeneous catalyst to ammonium chloride solution being 1:15. Subsequently, drying is effected at from 100 to 120° C.

A further modification which can be undertaken on aluminum-containing catalysts is a dealumination in which a portion of the aluminum atoms is replaced by silicon, or the aluminum content of the catalysts is depleted by, for example, hydrothermal treatment. A hydrothermal dealumination is followed advantageously by an extraction with acids or complexing agents, in order to remove nonlattice aluminum forms. Aluminum can be replaced by silicon, for example, with the aid of (NH₄)₂SiF₆ or SiCl₄. Examples of dealuminations of Y zeolites can be found in Cormm et al., Stud. Surf. Sci. Catal. 37 (1987), pages 495 to 503.

The modification by silylation is described in general terms in J. Weitkamp and L. Puppe, Catalysis and Zeolites, Fundamentals and Applications, Chapter 3: G. Kühl, Modification of Zeolites, Springer Verlag, Berlin, 1999. In general, the procedure is to block acidic sites selectively, for example the external sites with bulky bases, for example 2,2,6,6-tetramethylpiperidine or 2,6-lutidine, and then to treat the zeolite with suitable Si compounds, for example tetraethyl orthosilicate, tetramethyl orthosilicate, C₁-C₂₀-trialkylsilyl chloride, methoxide or ethoxide, or SiCl₄. This treatment may be effected either with gaseous Si compounds or with Si compounds dissolved in anhydrous solvents, for example hydrocarbons or alcohols. It is also possible to combine various Si compounds. Alternatively, the Si compound may also already contain the amine group selective for acidic sites, for example 2,6-trimethylsilylpiperidine. Afterward, the thus modified catalysts are generally calcined in O₂-containing atmosphere at temperatures of from 200 to 500° C.

A further modification consists in the blockage of external sites by mixing or grinding the catalyst powder with metal oxides, for example MgO, and subsequently calcining at from 200 to 500° C.

The heterogeneous catalysts are generally used in the form of extudates, spall or tablets having a characteristic diameter of from 0.1 to 5 mm, preferably from 0.5 to 3 mm. The characteristic diameter is calculated from six times the quotient of shaped body volume and geometric shaped body surface area.

The catalyst which can be used in the process according to the invention is activated by a reducing pretreatment.

It is carried out generally at a temperature of from 80 to 500° C., preferably at from 100 to 400° C., more preferably at from 150 to 300° C.

The reducing pretreatment is carried out, for example, with the aid of a gaseous or of a liquid reducing agent. It is possible to use all suitable reducing agents known to those skilled in the art for the reductive pretreatment of the catalyst; for example, hydrogen, inert gas-hydrogen mixtures, hydrogen-ammonia mixtures may be used. Alternatively, the reducing pretreatment, preferably by hydrazine, may be effected in the liquid phase.

The reductive pretreatment is carried out in a reactor suitable therefor which is known to those skilled in the art. It may also be effected in the reactor of the aromatic alkylation before the aromatic compounds and the alkanes are added.

### Preferred Reaction Procedure

The alkylation is carried out in such a way that the aromatic compound or the mixture of aromatic compounds and the alkane or the mixture of alkanes are allowed to react in a suitable reaction zone by contacting with the catalyst, working up the reaction mixture after the reaction and thus obtaining the products of value.

Suitable reaction zones are, for example, tubular reactors, stirred tanks or a stirred tank battery, a fluidized bed, a loop reactor or a solid-liquid moving bed. When the catalyst is in solid form, it may be used either in the form of a slurry, as a fixed bed, as a moving bed or as a fluidized bed.

When a fixed bed reactor is used, the reaction partners may be conducted either in cocurrent or in countercurrent. The configuration as a catalytic distillation is also possible.

The reaction partners are either in the liquid or in the gaseous state, but preferably in the liquid state. The reaction in the supercritical state is also possible.

The ratio of the reaction partners is selected such that, on the one hand, very substantial conversion of the alkane takes place and, on the other hand, very few by-products are formed. Possible by-products are in particular dialkylbenzenes, diphenylalkanes, polycyclic aromatics and alkane or olefin oligomers. The selection of the temperature also depends crucially upon the catalyst selected. Reaction temperatures between 20 and 500° C., preferably from 100 to 250° C., more preferably from 120 to 220° C., can be employed.

The pressure of the reaction depends upon the selected method (reactor type) and is from 1 to 200 bar, preferably from 1 to 50 bar, more preferably from 1 to 40 bar. The catalyst hourly space velocity (WHSV) is from 0.01 to 100, preferably from 1 to 10, more preferably from 0.1 to 5, g (reactant)/g (catalyst)*h.

The reaction partners may optionally be diluted in the gas phase/supercritical phase with inert substances. Preferentially suitable inert substances are perfluorinated alkanes, carbon dioxide, nitrogen, hydrogen and/or noble gases.

The process according to the invention may be carried out in substance or in solution. The reaction partners may be diluted in the liquid phase with solvents, Suitable solvents are, for example, the fluorinated alkanes, cyclic and/or linear ethers, or aromatic compounds; preference is given to using benzene.

The molar ratio between the aromatic hydrocarbon or the mixture of hydrocarbons and the alkane or the mixture of alkanes is from 100:1 to 1:100, preferably from 50:1 to 1:50, more preferably from 10:1 to 1:10.

The process according to the invention may be carried out batchwise, semicontinuously by initially charging, for example, catalyst and aromatic compound and metering alkane(s), or fully continuously, if appropriate also with continuous supply and removal of catalyst.

Catalysts having inadequate activities may be regenerated directly in the alkylation reactor or in a separate plant by
1. washing with solvents, for example alkanes, aromatics, for example benzene, toluene or xylene, ethers, for example tetrahydrofuran, tetrahydropyran, dioxane, dioxolane, diethyl ether or methyl t-butyl ether, alcohols, for example methanol, ethanol, propanol and isopropanol, amides, for example dimethylformamide, nitriles, for example acrylonitrile, or water, at temperatures of from 20 to 200° C.,
2. by treating with steam at temperatures of from 100 to 400° C.,
3. by thermally treating in a reactive gas atmosphere (O₂ and O₂-containing gas mixtures, CO₂, CO, H₂) at from 200 to 600° C. or
4. by thermally treating in an inert gas atmosphere (N₂, noble gases) at from 200 to 600° C., or
   by combinations of 1 to 4. Alternatively, deactivated catalyst, as described above, may also be added in the preparation of new catalyst.

The present invention also relates to a process for preparing alkylarylsulfonates by sulfonating and neutralizing the alkyl aromatic compounds which are obtained by the inventive reaction of aromatic compounds with C₁-C₁₄-alkanes. The sulfonation of the alkyl aromatic compounds may be effected by processes known to those skilled in the art. For example, the alkylaryls may be converted to alkylarylsulfonates by
1) sulfonating (for example with SO₃, oleum, chlorosulfonic acid, etc., preferably with SO₃) and
2) neutralizing (for example with Na, K, NH, Mg compounds, preferably with Na compounds).
   Sulfonation and neutralization are described sufficiently in the literature and are performed in accordance with the prior art. The sulfonation is preferably performed in a falling-film reactor, but may also be effected in a stirred tank, Sulfonation with SO₃ is to be preferred over sulfonation with oleum.

The following examples will illustrate the process according to the invention in detail:

### EXAMPLES

### Catalyst Preparation

### Example 1

480 g of BEA zeolite (H form) are mixed with 120 g of Plural SB and compacted in a kneader with 12 g of formic acid and 730 ml of deionized water. The catalyst mass is shaped to extrudates (diameter 2 mm) and dried at 120° C. for 16 hours. Subsequently, the thus obtained material is calcined at 500° C. for 16 hours. 600 g of the extrudates are impregnated with 600 g of a solution of hexachloroplatinic acid (0.5% by weight) and dried at 120° C. for 12 h. Subsequently, the catalyst is calcined at 500° C. for 5 h. For activation, the catalyst is initially predried at 80° C. in a nitrogen stream (100 L/h) for 30 min, and then the temperature is increased to 120° C. for a further 30 min. Subsequently, the temperature is increased slowly to from 180 to 200° C. and a mixture of 100 L/h of nitrogen and 5 L/h of hydrogen is metered in. Within 2 hours, the hydrogen fraction is increased to 50 L/h. Subsequently, the catalyst is activated in a pure hydrogen stream at 240° C. for 12 hours.

### Example 2

100 g of Y zeolite (H form, powder) are suspended in a tetraamineplatinum(II) hydroxide solution at 90° C. for 24 hours and subsequently filtered off. The operation is repeated twice more and the reaction effluent is dried at 120° C. for 12 hours. 100 g of the thus obtained material are mixed with 25 g of Plural SB and compacted in a kneader with 2.5 g of formic acid and 80 ml of deionized water. The catalyst mass is shaped to extrudates (diameter 2 mm) and dried in an air stream at 120° C. for 16 hours. Subsequently, the thus obtained material is calcined in an air stream at 500° C. for 16 hours. For activation, the catalyst is initially predried at 80° C. in a nitrogen stream (100 L/h) for 30 nm, and then the temperature is increased to 120° C. for a further 30 rain. Subsequently, the temperature is increased slowly to from 180 to 200° C. and a mixture of 100 L/h of nitrogen and 5 L/h of hydrogen is metered in. Within 2 hours, the hydrogen fraction is increased to 50 L/h. Subsequently, the catalyst is activated in a pure hydrogen stream at 240° C. for 12 hours.

### Example 3

250 g of BEA zeolite (H form, powder) are suspended in a tetraamineplatinum(II) hydroxide solution at 90° C. for 24 hours and subsequently filtered off. The operation is repeated twice more and the reaction effluent is dried at 120° C. for 12 hours. 22 g of the thus obtained material are mixed with 55 g of Plural SB and compacted in a kneader with 5.5 g of formic acid and 190 g of deionized water. The catalyst mass is shaped to extrudates (diameter 2 mm) and dried in an air stream at 120° C. for 16 hours. Subsequently, the thus obtained material is calcined in an air stream at 500° C. for 5 hours. For activation, the catalyst is initially predried at 80° C. in a nitrogen stream (100 Uh) for 30 min, and then the temperature is increased to 120° C. for a further 30 min. Subsequently, the temperature is increased slowly to from 180 to 200° C. and a mixture of 100 LA of nitrogen and 5 L/h of hydrogen is metered in. Within 2 hours, the hydrogen fraction is increased to 50 L/h. Subsequently, the catalyst is activated in a pure hydrogen stream at 240° C. for 12 hours.

Reaction of Alkanes with Benzene

A tubular reactor disposed in a forced-air oven was charged with 32 g of catalyst spall (from Examples 1-3) of particle size 0.7-1.0 mm and activated in a hydrogen stream at 200° C. for 24 h. Subsequently, the catalyst was baked at 250° C. for a further 6 h. The reactor was cooled to the operating temperature and pressurized to operating pressure 30 bar with a feed composed of dodecane and benzene (1:10 molar). The reactor was operated with backmixing. To this end, an approx. tenfold higher circulation stream relative to the feed was established.

The content of reactants and products in the effluent stream was detected by means of time-resolved GC and online IR. The resulting C₁₈-alkylaryl mixture was purified by distillation and analyzed by means of gas chromatography-mass spectrometry coupling and ¹H/¹³C NMR spectroscopy. LAB means linear alkylbenzene. The results are compiled in Table 1.

TABLE 1

| | | | | Hourly | |
|---|---|---|---|---|---|
| | Catalyst | | | space | LAB |
| Example | from | Feed | Temp. | velocity | [% by |
| No. | example | (benzene:dodecane) | [° C.] | [g/gh] | wt.] |
| 4 | 1 | 4:1 | 160 | 0.3 | 0.1 |
| 5 | 1 | 4:1 | 180 | 0.3 | 2.2 |
| 6 | 1 | 4:1 | 180 | 0.6 | 1.3 |
| 7 | 1 | 4:1 | 185 | 0.6 | 1.6 |
| 8 | 1 | 4:1 | 190 | 0.6 | 2.0 |
| 9 | 1 | 4:1 | 195 | 0.6 | 2.4 |
| 10 | 1 | 4:1 | 195 | 1.2 | 1.2 |
| 11 | 2 | 4:1 | 180 | 0.3 | 0.2 |
| 12 | 2 | 4:1 | 190 | 0.3 | 0.6 |
| 13 | 2 | 4:1 | 200 | 0.3 | 0.9 |
| 14 | 2 | 4:1 | 210 | 0.3 | 1.3 |
| 15 | 2 | 4:1 | 220 | 0.3 | 1.9 |
| 16 | 2 | 4:1 | 230 | 0.3 | 2.0 |
| 17 | 2 | 4:1 | 240 | 0.3 | 2.2 |
| 18 | 3 | 4:1 | 180 | 0.3 | 3.8 |
| 19 | 3 | 4:1 | 185 | 0.3 | 4.0 |
| 20 | 3 | 4:1 | 190 | 0.3 | 4.3 |
| 21 | 3 | 4:1 | 190 | 0.6 | 3.4 |

Tables 2 and 3 summarize the results of the comparative experiments.

TABLE 2
Comparative examples, a catalyst
was used without preceding doping

| | | | | Hourly | |
|---|---|---|---|---|---|
| | | | | space | LAB |
| Example | | Feed | Temp. | velocity | [% by |
| No. | Catalyst | (benzene:dodecane) | [° C.] | [g/gh] | wt.] |
| 22 | H-BEA | 4:1 | 160 | 0.3 | 0.4 |
| 23 | H-BEA | 4:1 | 180 | 0.3 | 0.4 |
| 24 | H-BEA | 4:1 | 200 | 0.3 | 0.4 |
| 25 | H-BEA | 4:1 | 220 | 0.3 | 1.0 |
| 26 | H-BEA | 4:1 | 240 | 0.3 | 1.2 |

TABLE 3
Comparative examples, a catalyst without
preceding activation was used

| | | | | Hourly | |
|---|---|---|---|---|---|
| | Catalyst | | | space | LAB |
| Example | from | Feed | Temp. | velocity | [% by |
| No. | example | (benzene:dodecane) | [° C.] | [g/gh] | wt.] |
| 27 | 2 | 4:1 | 180 | 0.3 | 0.1 |
| 28 | | 4:1 | 190 | 0.3 | 0.3 |
| 29 | 2 | 4:1 | 200 | 0.3 | 0.5 |
| 30 | 2 | 4:1 | 210 | 0.3 | 0.8 |
| 31 | 2 | 4:1 | 220 | 0.3 | 1.1 |
| 32 | 2 | 4:1 | 230 | 0.3 | 1.3 |
| 33 | 2 | 4:1 | 240 | 0.3 | 1.5 |

Under the above-outlined conditions of the reaction of benzene with dodecane, other alkanes may also be reacted with benzene. The results achieved are compiled in Table 4.

TABLE 4

| | | | | Hourly | |
|---|---|---|---|---|---|
| | Catalyst | | | space | LAB |
| Example | from | Feed | Temp. | velocity | [% by |
| No./alkane | example | (benzene:alkane) | [° C.] | [g/gh] | wt.] |
| 34/cyclohexane | 1 | 2:1 | 180 | 0.3 | 3.7 |
| 35/ethane | 1 | 2:1 | 180 | 0.3 | 4.0 |
| 36/propane | 1 | 2:1 | 180 | 0.3 | 2.5 |

1-9. (canceled)

10. A process for preparing alkylaromatics by reacting aromatic compounds with C₁-C₁₄-alkanes in the presence of a heterogeneous catalyst, which comprises using as the catalyst a crystalline, micro- and/or mesoporous solid having average pore radii of up to 2 nm and/or from 2 to 50 nm comprising silicon and at least one further element selected from the group consisting of the transition metals and the main group elements gallium and tin, and activating said catalysts by a reducing pretreatment.

11. The process according to claim 10, wherein the catalyst comprises, as a further element, an element selected from the group consisting of the elements of groups 6, 7, 8, 9, 10, 11, cerium, zinc, lanthanum and zirconium.

12. The process according to claim 10, wherein a C₉-C₁₄-alkane is used.

13. The process according to claim 12, wherein a C₁₀-C₁₃-alkane is used.

14. The process according to claim 10, wherein the catalyst is at least one zeolite or at least one clay.

15. The process according to claim 14, wherein the zeolite is selected from the group of structural classes consisting of FAU, MOR, BEA, MFI, MEL, TON, MTW, ZBM-11, PER, LTL, MAZ, EPI, GME.

16. The process according to claim 10, wherein monocyclic aromatic compounds are used.

17. The process according to claim 16, wherein the aromatic compound is selected from the group consisting of benzene, toluene, ethylbenzene and the isomers of xylene.

18. A process for preparing alkylarylsulfonates by sulfonating and neutralizing the alkyl aromatic compounds prepared by a process according to claim 10.
